(19) United States
(12) Patent Application Publication
(10) Pub. No.: US 2006/0280695 A1
(43) Pub. Date: Dec. 14, 2006

(54) Methods and compositions for the prevention, suppression and elimination of oral pain

(76) Inventor: Brian C. Giles, Montecito, CA (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/149,824

(22) Filed: Jun. 8, 2005

(51) Int. Cl. A61K 8/19

(52) U.S. Cl. 424/49

(57) ABSTRACT

The present invention relates to methods and compositions for the prevention, suppression and elimination of oral pain. Specifically, it relates to methods and compositions including cesium and/or rubidium that are used to treat or prevent oral pain. In a method aspect of the present invention, a method of treating oral pain in a mammal is provided. The method involves topically administering or injecting a composition to the oral cavity of the mammal, and wherein the composition comprises from 500 ppm to 100,000 ppm of a cesium ion source, a rubidium ion source, or a mixture of the two.

### FIELD OF THE INVENTION

The present invention relates to methods and compositions for the prevention, suppression and elimination of oral pain. Specifically, it relates to methods and long-acting compositions including cesium and/or rubidium that are used to treat and eliminate the primary source of pain and its signaling.

### BACKGROUND OF THE INVENTION

Oral pain is a serious problem for many individuals, and it takes on a variety of guises. It may be associated, for example, with ulcers, canker sores, teething, oral cancer and its treatments, or with dental procedures, such as, but not limited to, surgical procedures, teeth cleaning, gum cleaning and teeth scraping. This limitation has been readily discerned by the medical industry, which has marketed a host of products aimed at oral pain.

ANBESOL®, for instance, is an over the counter composition marketed for oral pain. It is traditionally, topically applied to reduce pain resulting from teething, canker sores, and denture irritation. PEROXYL® Antiseptic Dental Rinse is marketed to promote oral wound healing and to reduce resulting pain. ORABASE® is used to provide temporary relief of pain created by braces or dentures. BONJELA® Oral Pain-Relieving Gel is applied to relieve teething and mouth ulcer pain. And, lignocaine- and xylocaine-based rinses are sold for the treatment of oral mucositis symptoms.

Local anesthetics are also used for the prevention of oral pain resulting from dental procedures. Compounds such as benzocaine are formulated for either topical application or injection and are directly applied to areas that will be the subject of a procedure. The formulations prevent and/or treat pain by reversibly blocking the action potentials responsible for nerve conduction.

There is a need for further compositions and methods that can be used to treat or prevent a wide variety of oral pain. That is an object of the present invention.

### VARIOUS REFERENCES

“Changes in Tissue pH and Temperature after Incision Indicate Acidosis May Contribute to Postoperative Pain.” Abstract: Anesthesiology. Volume 101 (2) August 2004 p. 468-475.

S. Kalfas et al., Effect of pH on acid production from sorbitol in washed cell suspensions of oral bacteria, Caries Res, 24(2):107-12 (1990)—Abstract Only.

P. L. Schroeder et al., “Dental erosion and acid reflux disease,” Ann Intern Med, 122(11):809-15 (Jun. 1, 1995).

D. C. Abelson et al., “The effect of saliva on plaque pH in vivo,” J Dent Res, 60(9):1634-8 (September 1981)—Abstract Only.

F. M. Eggert et al., “The pH of gingival crevices and periodontal pockets in children, teenagers and adults,” Arch Oral Biol, 36(3):233-8 (1991)—Abstract Only.

G. Maglis et al., “Determination of saliva pH in periodontal disease patients and a control group,” Rev Dent Chile, 80(2):70-2 (August 1989)—Abstract Only.

I. D. Mandel, “The role of saliva in maintaining oral homeostasis,” Journal of the American Dental Association, 8(8:298 (7) (1989)—Abstract Only.

J. R. Newland, “Oral ulcers: keys to differential and definitive diagnosis,” Consultant, 29(5):157(11) (May 1989).

Lyda Associates Inc..RTM. 1990, “Dental properties of soft drinks,” Nutrition Research Newsletter, 9(1:7(1) (January 1990).

C. C. Schurer-Maly et al., “Smoking and pH response to H2-receptor antagonists,” Scand J Gastroenterol, 24(10):1172-8 (December 1989)—Abstract Only.

L. A. Elson et al., “The Sugar Content And The pH Of The Smoke Of Cigarette, Cigar And Pipe Tobaccos In Relation To Lung Cancer,” Int J Cancer, 9(3):666-675 (1972)—Abstract Only.

C. J. Thomas et al., “Astringent subqualities in acids,” Chem Senses, 20(6):593-600 (December 1995)—Abstract Only.

The American Dietetic Association.RTM., Single Information Sheet, “FIG. 1. Definitions of oral health terms,” (Mar. 19, 1997).

News Bites, Single Sheet, “Sports Drinks: Bad for Teeth?” HealthNews, p. 8 (Apr. 15, 1997).

Single page, “Medical Update—Heartburn and Tooth Trouble,” Consumer Reports on Health, p. 106 (September 1995).

T. H. Grenby, “Comparison of the Cariostatic Effects of Calcium and Sodium Glycerophosphates in Rats,” Helv. Odont. Acta, 17:54, 55 (October 1973).

A. H. Brook et al., “A Clinical Study of the Effect of Calcium Glycerophosphate,” Helv. Odont. Acta, 17:55 (October 1973).

P. Vahl et al., “Examination of the pH of saliva in children and juveniles in relation to caries, gingivitis and oral hygiene,” Stomatol DDR, 39(4):253-8 (April 1989).

Stehpan, Megan M., “Signals from the Frontlines,” The Scientist, Mar. 28, 2005.

### SUMMARY OF THE INVENTION

The present invention relates to methods and compositions for the prevention, suppression and elimination of oral pain. Specifically, it relates to methods, treatments and compositions including cesium and/or rubidium that are used to treat or prevent oral pain.

In a method aspect of the present invention, a method of treating oral pain in a mammal is provided. The method administration by either topical administration to or injection in the oral cavity of the mammal, and wherein the composition comprises from 500 ppm to 100,000 ppm of a cesium ion source, a rubidium ion source, or a mixture of the two.

In another method aspect of the present invention, a method of anesthetizing oral tissue of a mammal is provided. The method administration by either topical administration to or injection in the oral cavity of the mammal, and wherein the composition comprises from 500 ppm to 100,000 ppm of a cesium ion source, a rubidium ion source, or a mixture of the two.

In a composition aspect of the present invention, a composition comprising from 500 ppm to 100,000 ppm of a cesium ion source, a rubidium ion source, or a mixture of the two; and water having a surface tension ranging from 30 to 70 dynes per cm² is provided.

### DETAILED DESCRIPTION OF THE INVENTION

Definitions

The terms “acidity” and “alkalinity” refer to values measured by pH, which is defined as the negative logarithm of the Hydrogen ion activity: pH=−log(H+). The parameter pHe is the pH on the exterior and pHi is the pH on the interior of the cell.

The term “acid” refers to a substance consisting of molecules of ions which donate protons (H+), and a “base” is a substance which accepts protons.

The term “acid-forming reaction” refers any chemical reaction that produces a decreased ability to energize the biological system and leaves an acid residue, such as a hydrogen ion (H+). The result is localized acidosis with induced hypoxia, the major cause of a wide varieties of oral pain.

The term “reducing acidity” or “to reduce the acidity of” as used herein in relation to the intra-oral tissues, mouth or the throat of a mammal means to raise and/or maintain the pH, pHe and pHi of a surface tissues or a portion of the mouth, throat or saliva therein such the various pH's are above the action potentials responsible for nerve conduction (i.e., 6.80 or higher). The surface or portion of the mouth or the throat or the saliva therein may include, by way of example and not by way of limitation, the surface tissues or a portion of the teeth, the gums, the tongue, and the back of the throat of a mammal.

The term “mammal” has its normal dictionary meaning. Specific examples of mammals include humans, dogs, cats, horses, and cows.

Introduction

Oral health can be influenced by many factors. One of the most fundamental factors is the correct function of homeostatic regulation of ionic concentrations, pH and membrane electrical potentials. It is currently known that the biochemical activities of living cells are controlled by pH and ion concentrations; many key processes, such as energy metabolism and nerve and brain function, depend on membrane electrical potential. It is also known that ion movements, correct pH and membrane electrical potential are closely linked through the action of ion specific gates, electrogenic carriers and other ion carriers in cell and organelle membranes.

Most oral degenerative diseases, including pathogen invasion and propagation, are caused by a reduction in the intra-oral pH, pHe and pHi and progress though a complex series of pathological and electrochemical changes from initial onset to full manifestation. Unfortunately, drugs currently on the market do not address the complexity of acidosis, hypoxia and inflammation disease processes, the resultant pain and the underlying source, causes or mechanisms. This oftentimes makes them difficult to understand without knowing the electro-chemical and electro-physiological mechanisms.

There are many drugs and therapies aimed at correcting specific disease symptoms including pain that result from failure to maintain the optimum or near optimum pH range and the functional ionic physiological conditions. Preventative protection of the biological function from failure of homeostasis has generally been relegated largely to the realm of nutritional and lifestyle choices. The knowledge and understanding of ionic physiology enable us to provide oral formulations and methods that will make the protection of ionic homeostasis, such as minimizing the accumulation of acidic and hypoxia damage and the resultant pain within the tissues and cells, etc., accessible and convenient, resulting in the prophylaxis and elimination of pain.

An acidic biological environment that promotes the pain signal (reduced pH) may be caused by at least two situations: 1) where reductant to the electron transport chain exceeds electron acceptor capacity; and, 2) where an incorrect electro-physical state occurs in H+ or other ion gradients due to metabolic dysregulation. The targeted “increase” of pH and pHe in oral cells and tissues to reach a physiologically optimum range provides resistance to a wide variety of secondary infections. Such a response is instrumental in pain reduction and elimination.

The aberrant regulation of cellular homeostasis is a significant factor in the pathogenesis of disease onset. Raising the pH of the oral region, cellular pHe and cellular pHi reduces the excessive excitement of neurons, processes the stressful biological inflammatory complex free radical oxidative stress (such as super oxides, peroxides, oxyacids, alcohols and aldehydes), restores and stabilizes the ionic homeostasis, processes enzyme toxins and releases the useful molecular oxygen from its bound state. As an example, the central disorders of acute maladaptive reactions (pain) are oxygen deficit and acidity. The biochemistry of chronic and acute degenerative diseases reveals the same disorders as acute maladaptive reactions with a sub optimal pH that produces the acidic biological environment signaling pain.

Cesium and/or Rubidium ions provide an “electron bath.” The intra-oral tissues are bathed in electrons, and they are then stabilized and no longer able to induce cellular tissue damage and signaling pain. The optimally functioning oral electrochemical environment has a narrow pH ranging from 7.00 to 7.55, preferably between 7.30 and 7.45. The method and formula provide for oral pH increase and obtaining a reduction of the H+ migration and other fluxes, providing resistance to oral pain by producing ionic changes in the pH, pHe, pHi, and changes the ionic chemistry of the intra-oral cells and tissues.

### OBJECTS AND ADVANTAGES OF THE INVENTION

An advantage of the present invention is that it prevents and eliminates the formation of an intra-oral, acidic or hypoxic or inflammatory electrochemical environment, which minimizes the damage within cells and tissues.

A further advantage of the present invention is that it can be cost effectively administered as a stand alone therapy or as an effective adjunct in conjunction with a wide variety of known therapies.

An object of the present invention is to provide a composition that can prevent, suppress or eliminate oral pain upon topical application or injection.

A further object of the present invention is to provide a method by which oral pain is treated or prevented.

The present invention provides an alkaline, ionic, pH manipulating formula and therapy for inhibiting acidotic activity (reduced pH) for the prevention, suppression and elimination of oral pain. The therapy involves the administration of sufficient quantities of alkaline salts, in a suitable carrier, or in a wide variety of delivery forms that provide for optimum or near optimum ionic, pH, electrochemical and electro-physiological requirements. Specifically, the treatment of mammals, and more specifically human patients, involves the administration of a therapeutically effective dose of a salt or salts of cesium and/or rubidium, optionally with supportive electrolytes, vitamins, trace minerals and other nutrients.

Cesium and rubidium are the two most alkali metals with chemical and physical characteristics similar to potassium. Potassium is the main internal cation of living cells. Potassium ion currents are central to the ionic physiology of normal healthy viable cells. Trans-membrane fluxes and cellular accumulation of cesium and rubidium ions are governed-by the similar cellular mechanisms as those which govern potassium movements; however, cesium and rubidium ions move at slower rates and accumulate to different degrees. Cesium and rubidium ions are effective for the control of potassium fluxes and linked hydrogen ions and other ionic fluxes.

Delivery of Compositions

The compositions/formulations of the present invention may be administered by an acceptable route. Typically, the compositions/formulations are applied topically as a rinse, gel, paste, lozenge, gum, floss or spray. Another route of application is through the application of a surgical bandage or a swab, where the composition is impregnated within the bandage or swab. A further route is through direct injection of the formulation/composition into a subject tissue.

Compositions

Preferably, the oral compositions of the present invention include a cesium or rubidium ion source present in an amount sufficient to provide from 500 ppm to 100,000 ppm, preferably 1,000 ppm to 10,000 ppm, cesium and/or rubidium.

Any combination of cesium and/or rubidium salts which dissociate and ionize may be employed in the composition of the present invention, including, but not limited to: Arginate, Ascorbate, Aspartate, Caprylate, Chloride, Cysteinate, Citrate, Fumarate, Humic, Fulvate, Methionine, Glutamate, Gluconate, Glycinate, Aspartate, Lysinate, Succinate, Carbonate, Lactate, Malate, Tartrate, Sulfate, Phosphate, Nitrate, Flouride, Bromide, Iodide, Orotate, Asporotate, Bisulfonate, Lysinate, Fulvic, Succate, Carnate, Trisulfate, Lactobionate, Benzenesulfonate, Laurate, Benzoate, Bicarbonate, Benzoic, Caseinate, Bisufate, Mandelate, Bitartrate, Mesylate, Borate, Methylbromide, Methylnitrate, Calcium Edetate, Methylsulfate, Camsylate, Mucate, Napsylate, Clavulanate, N-Methylglucamine, Ammonium Salt, Dihydrochloride, Oleate, Edetate, Oxalate, Edisylate, Pamoate (Embonate), Esolate, Palmitate, Esylate, Pantothenate, Phosphate/Diphosphate, Gluceptate, Polygalacturonate, Salicylate, Stearate, Glycollylarsanilate, Hexylresorcinate, Subacetate, Hydrabamine, Hydrobromide, Tannate, Hydrochloride, Hydroxynaphthoate, Teoclate, Tosylate, Isothionate, Triethiodide, Panoate, Valerate, Acetate, Maleate, Malonate, Sulfate, and mixtures thereof.

Additionally, other cesium and rubidium salts might be used in a wide variety of intra-oral compositions, such as, but not limited to, various organic or metallic salts, if they meet the following requirements: (1) they must be pharmaceutically acceptable and have an acceptably low level of toxicity; (2) they must have sufficient high levels of cationic (alkaline) dissociation to allow the remaining negatively charged ions to effectively reduce the intra-oral cavity acidity, including tissues to reduce or eliminate pain.

The salts included in the composition of the present invention may be formed using a wide variety of acids, including, but not limited to: hydrochloric, carbonic, humic, fulvic, sulfuric, acetic, lactic, tartaric, malic, succinic, etc. Malic acid, hydrochloric acid, carbonic acid and citric acid are preferred acids.

Potentiation of cesium and/or rubidium ionic action can be accomplished by inclusion of ingredients that enhance ionic pH physiology. Examples are electrolytes (saline compounds) such as potassium, sodium, and magnesium. Potassium, and other major electrolytes (e.g., sodium, calcium, chloride, bicarbonate, phosphate and sulfates) are added to the formulation in proportion to the potassium.

Other ingredients that may be included to potentiate the activity of cesium/rubidium ionic action include manganese, zinc, boron, vitamin B2 (riboflavin), B12 (cyanocbalamin), and B6 (pyridoxine).

A solvent (e.g., water), especially in the case of a rinse, spray or injectable solution, may be included in the composition. Where the solvent is water, a preferred method of manufacturing is to use water processed by means such as E.C.A. (electrolytic chemical activation) processing. The method produces aqueous solutions having certain characteristics. For example, an aqueous rinse solution (water) for an oral treatment may be processed having a surface tension ranging from 30 to 70 dynes per cm², preferably ranging from 40 to 60 dynes per cm² with an ORP (oxidative reduction potential) ranging between −5 m.v. to −400 m.v., preferably ranging between −10 m.v. to −250 m.v. (after dissociation). The pH for such compositions ranges from 6.40 to 8.50, preferably 6.80 to 8.40.

In a further example, an aqueous injection solution (water) for an oral treatment may be processed having a surface tension ranging from 50 to 70 dynes per cm², with an ORP (oxidative reduction potential) ranging between −200 m.v. to −400 m.v., preferably ranging between −220 m.v. to −280 m.v. (after dissociation). The pH for such compositions ranges from 7.20 to 7.50, preferably 7.30 to 7.40.

The compositions may contain the following substances, if they do not interfere with the dissociation and mobility of alkaline ions: humectants, gelling agents, abrasives, fluoride sources, desensitizing agents, flavorings, colorings, sweeteners, preservatives and structuring agents, surfactants, anti-calculus agents and anti-plaque agents. Suitable surfactants are water-soluble organic compounds, and may be nonionic, cationic or amphoteric species throughout a suitable pH range.

Structuring agents may be used in, for example, dentifrices and gums to provide desirable textural properties and “mouth feel”. Suitable agents include natural gum binders such as gum tragacanth, xanthan gum, gum karaya and gum arabic, seaweed derivatives, smectite clays such as diatomaceous earths, bentonite or hectorite, calcium apatite, carboxyvinyl polymers and water-soluble cellulose derivatives such as hydroxyethyl cellulose and sodium carboxymethyl cellulose. Improved texture may also be achieved, for example, by including colloidal magnesium and or aluminum silicate. Suitably, the structuring agent is included in an amount of from 0-5%, preferably 0-3% by weight of the composition.

Abrasive agents including silica abrasives, such as hydrated silicas and silica gels, particularly silica xerogels. Alternative abrasives include alumina, insoluble metaphosphates such as insoluble sodium metaphosphate, calcium carbonate, dicalcium phosphate (in dihydrate and anhydrous forms), and calcium pyrophosphate (including beta-phase calcium). Calcium carbonate is a preferred abrasive. Abrasives are typically included in an amount of from 0-80%, preferably 0-60%, more preferably 5-25% by weight of the oral hygiene composition. The abrasives typically have an average particle size of 0.1-30 microns, preferably 5 to 15 microns.

Flavoring agents may be included in the oral composition if desired, as long as they do not substantially interfere with the ionic dissociation and mobility of cesium and/or rubidium. Such agents—e.g., oils of citrus, lemon-lime, spearmint, menthol, cherry, wintergreen, sassafras and clove—are typically included to increase the palatability of a composition.

Flavoring agents may be lipophilic or hydrophilic. Lipophilic flavorants include, without limitation, wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavender oil, mustard oil, pine oil, pine needle oil, rosemary oil, thyme oil, cinnamon leaf oil, and mixtures thereof. Where used, lipophilic flavorants are typically included in the oral composition at a level from 0.01%-10% by weight, preferably from 0.05%-5.0% by weight, and more preferably from 0.1%-3.0% by weight.

Sweetening agents such as the following may also be included: D-tryptophan, dextrose, levulose, acesulfam, dihydrochalcones and sodium cyclamate. Such flavoring or sweetening agents are typically included in the composition in an amount from 0-5% by weight, preferably 0-2% by weight. Furthermore, coloring agents (e.g., colorants or pigments) may be added to improve the visual appearance of the composition. Suitable colorants include, without limitation, dyes such as FD & C blue No. 1, D & C yellow No. 10 and D & C yellow No. 3. A suitable and commonly used pigment is titanium dioxide, which provides a white color.

Other optional components for use in the present compositions include: antioxidants; vitamins (e.g., vitamin C and E); other anti-plaque agents (e.g., stannous salts, copper salts, and magnesium salts); pH adjusting agents; anticaries agents (e.g., urea, calcium glycerophosphate, and sodium trimetaphosphate), and plant extracts.

The compositions of the present invention may further optionally include a traditional local anesthetic. Examples of such local anesthetics include, without limitation, the following: benzocaine; bupivacaine; etidocaine; lidocaine; mipivacaine; pramoxine; prilocaine; procaine; proparacaine; ropivacaine; and, tetracaine. Preferred local anesthetics are lidocaine. The local anesthetics are typically present from 0.1 percent by weight to 20 percent by weight of the composition; preferably they are present from 0.1 percent by weight to 5 percent by weight.

Where the composition of the present invention is used as a mouth rinse, it is preferred that the ingredients of the aqueous solution are selected such that the composition may be ingestible, even by children. A non-limiting example is where the aqueous solution is free of alcohol or other active ingredients that warrant poison control labeling or hazard labeling indicating that the composition is to be kept away from children. In a preferred mouth rinse embodiment, the ingredients of the rinse composition do not include alcohol, cetylpyridinium or witch hazel.

Exemplary Compositions

The following compositions are examples of compositions of the present invention:

Composition 1, which comprises the following: from 500 ppm to 100,000 ppm cesium ion source; and, water having a surface tension ranging between 30 and 70 dynes per cm².

Composition 2, which comprises the following: from 500 ppm to 100,000 ppm rubidium ion source; and, water having a surface tension ranging from 30 to 70 dynes per cm², preferably 40 to 60 dynes per cm².

Composition 3, which comprises the following: from 500 ppm to 100,000 ppm cesium ion source; a structuring agent included in an amount from 0.01 to 5 weight percent.

Composition 4, which comprises the following: from 500 ppm to 100,000 ppm rubidium ion source; a structuring agent included in an amount from 0.01 to 5 weight percent.

Composition 5, which comprises the following: from 500 ppm to 100,000 ppm cesium ion source; an abrasive included in an amount from 5 to 25 weight percent.

Composition 6, which comprises the following: from 500 ppm to 100,000 ppm rubidium ion source; an abrasive included in an amount from 5 to 25 weight percent.

Composition 7, which comprises the following: from 500 ppm to 100,000 ppm cesium ion source; a flavoring agent included in an amount from 0.1 to 5 weight percent.

Composition 8, which comprises the following: from 500 ppm to 100,000 ppm rubidium ion source; a flavoring agent included in an amount from about 0.1 to 5 weight percent.

Composition 9, which comprises the following: from 500 ppm to 100,000 ppm cesium ion source; potassium nitrate included in an amount from 2 to 10 weight percent.

Composition 10, which comprises the following: from 500 ppm to 100,000 ppm rubidium ion source; potassium nitrate included in an amount from 2 to 10 weight percent.

Composition 11, which comprises the following: from 500 ppm to 100,000 ppm cesium ion source; menthol included in an amount from 0.2 to 1.0 weight percent.

Composition 12, which comprises the following: from 500 ppm to 100,000 ppm rubidium ion source; menthol included in an amount from 0.2 to 1.0 weight percent.

Composition 13, which comprises the following: from 500 ppm to 100,000 ppm cesium ion source; phenol included in an amount from 0.2 to 1.0 weight percent.

Composition 14, which comprises the following: from 500 ppm to 100,000 ppm rubidium ion source; phenol included in an amount from 0.2 to 1.0 weight percent.

Composition 15, which comprises the following: from 500 ppm to 100,000 ppm cesium ion source; and oil of clove.

Composition 16, which comprises the following: from 500 ppm to 100,000 ppm rubidium ion source; and oil of clove.

Methods of Treatment

The formula and method will eliminate the intra-oral acidification (the source of pain) so that the physiologic pH is restored and approaches optimal, or near optimal ranges from 7.00 to 7.50, preferably 7.32 to 7.44. If pHe is close to optimum physiologic levels, pain is eliminated, metabolic function is restored and cellular regeneration and repair takes place.

Compositions of the present invention, such as those discussed above, may be used to prevent, suppress or eliminate oral pain. The compositions are used to prevent pain, for example, when they are used as local anesthetics. This use is typically performed before a dental procedure. In this case the compositions are usually either applied topically (e.g., as a rinse, paste, gel, impregnated swab, dressing or bandage) or are injected directly into the site of the procedure.

There are a number of situations where the compositions are used to suppress or eliminate oral pain. When a patient has a sore in his/her mouth, such as a canker sore or ulcer, application of compositions of the present invention will lessen or even completely eliminate associated pain. Application to sores oftentimes takes the form of a rinse, gel or lozenge. Oral procedures such as teeth cleaning and surgery damage gum tissue, which leads to pain and soreness. Use of the present compositions lessens or eliminates the pain resulting from such procedures.

Teething is another process that leads to severe discomfort and pain, especially in infants. The present compositions may be topically applied (e.g., paste, gel or liquid) to alleviate the symptoms. Denture wearers further suffer pain and soreness resulting from improper fit or wear. Relief from such pain can be obtained through application of the subject compositions.

Many people suffer from tooth sensitivity (i.e., dentin hypersensitivity), which is typically experienced as discomfort or pain after eating hot or cold foods or after breathing in cold air. The present compositions may be applied directly to sensitive teeth to reduce or eliminate pain or discomfort. The relief offered by the compositions is typically long-lasting, such that the reduction or elimination in pain occurs for a substantial period of time.

Oral mucositis, also called stomatitis, is a debilitating complication of chemotherapy and radiotherapy, which occurs in 40 percent of cancer patients. It consists of oral inflammation of the mucosa of the mouth and ranges from redness to severe ulceration—a condition that is at least uncomfortable and at most severely painful. Compositions of the present invention may be used to treat the pain and discomfort associated with these conditions.

Treatment of oral pain using the compositions and methods of the present invention is not limited to humans. Domestic animals, such as dogs, cats, and horses, suffer from oral discomfort and pain, and they may be treated with the subject compositions.

The optimally effective formula and dosage(s) may be adjusted (increased or decreased) as therapy progresses. A patient's saliva pH should be monitored during the treatment process and the dosage appropriately adjusted. The goal of dosage adjustment is to partially or wholly restore and maintain the physiologic optimum oral pH range from 7.00 to 7.55, more preferably from 7.30 to 7.50, and the oral cellular pHi above 6.40, preferably from 6.50 to 6.80, more preferably from 6.70 to 6.80.

Cesium and rubidium ions used in the present are separate and distinct from man-made isotopes of cesium and rubidium.

### EXAMPLE 1

A sixty year old male having active periodontal disease (stage II and localized stage III) took a composition into his mouth, swished it around for approximately 20 to 30 seconds and expelled it twice daily for a period of about 2 months. The composition included cesium chloride (30 mg), rubidium chloride (10 mg), potassium citrate (350 mg) and magnesium ascorbate (20 mg) in 6 to 8 ounces of warm water. The treatment resulted in substantial suppression and elimination of the patient's periodontal disease. Inflammation and bleeding within gingival tissues were eliminated, and plaque deposits went from being moderate to very slight.

### EXAMPLE 2

An eighty-two year old female having painful ulcers on her tongue and gums (8 to 12 millimeters) for over a year took a composition into her mouth, swished it around for approximately 20 to 30 seconds and expelled it twice daily. She experienced no pain for 12 to 15 hours. The composition included CsCl (50 mg), potassium (110 mg), calcium (25 mg), magnesium (30 mg), manganese (0.5 mg) and chromium (5 mcg) in 6 to 8 ounces of warm water. After 4 days, ulceration on her tongue and gums disappeared.

### EXAMPLE 3

A forty-seven year old male having painful swollen and bleeding gums and loose teeth took a composition into his mouth, swished it around for approximately 20 to 30 seconds and expelled it 2 to 3 times daily. (The gum problem had persisted for more than 20 years.) The composition included cesium chloride (15 mg), rubidium chloride (5 mg), potassium citrate (175 mg) and magnesium ascorbate (10 mg) suspended in 6 to 8 ounces of warm water. After 4 days, the patient could reinsert a dental bridge that had been previously uninsertable; after 8 days, all gum swelling, gum bleeding and tooth looseness disappeared. (Testing saliva pH pretreatment resulted in a reading of approximately 5.30 to 5.50; it was between 7.10 to 7.30 seven days post treatment.)

### EXAMPLE 4

A 50 year old male subject having just cracked a tooth began experiencing pain. The subject immediately took a composition into his mouth, swished it around for approximately 30 seconds and expelled it. The composition included CsCl (50-60 mg) and a drop of malic acid in 3 to 6 ounces of warm water. The subject's pain was almost immediately was eliminated, and the pain elimination lasted for a period of approximately 12 to 16 hours.

1. A method of treating oral pain in a mammal having an oral cavity, wherein the method comprises topically administering a composition to the oral cavity of the mammal, and wherein the composition comprises from 500 ppm to 100,000 ppm of a cesium ion source, a rubidium ion source, or a mixture of the two.

2. The method according to claim 1, wherein the composition comprises from 500 ppm to 100,000 ppm of a cesium ion source.

3. The method according to claim 1, wherein the composition comprises water having a surface tension ranging between 30 and 70 dynes per cm².

4. The method according to claim 1, wherein the composition comprises from 1,000 ppm to 10,000 ppm of a cesium ion source, a rubidium ion source, or a mixture of the two.

5. The method according to claim 1, wherein the composition is in the form of a spray, a rinse, a lozenge, gel, bandage, swab, or a paste.

6. A method of anesthetizing oral tissues of a mammal, wherein the method comprises topically administering a composition to the oral region and tissue, and wherein the composition comprises from 500 ppm to 100,000 ppm of a cesium ion source, a rubidium ion source, or a mixture of the two.

7. The method according to claim 6, wherein the composition comprises from 500 ppm to 100,000 ppm of a cesium ion source.

8. The method according to claim 6, wherein the composition comprises water having a surface tension ranging between 30 and 70 dynes per cm².

9. The method according to claim 6, wherein the composition comprises from 1,000 ppm to 10,000 ppm of a cesium ion source, a rubidium ion source, or a mixture of the two.

10. The method according to claim 6, wherein the composition further comprises a traditional local anesthetic.

11. A composition comprising:
a) from 500 ppm to 100,000 ppm of a cesium ion source, a rubidium ion source, or a mixture of the two; and,
b) water having a surface tension ranging between 30 and 70 dynes per cm².

12. The composition according to claim 11, wherein the composition further comprises benzocaine or lidocaine.

13. The composition according to claim 11, wherein the composition further comprises potassium nitrate.

14. The composition according to claim 11, wherein the composition comprises from 1,000 ppm to 10,000 ppm of a cesium ion source.
